(19) United States
(12) Patent Application Publication
(10) Pub. No.: US 2013/0190799 A1
(43) Pub. Date: Jul. 25, 2013

(54) Occluding Device

(71) Applicant: Boston Scientific Scimed, Inc., Maple Grove, MN (US)

(72) Inventor: Christopher J. Clark, St. Michael, MN (US)

(73) Assignee: Boston Scientific Scimed, Inc., Maple Grove, MN (US)

(21) Appl. No.: 13/746,660

(22) Filed: Jan. 22, 2013

Related U.S. Application Data
(60) Provisional application No. 61/589,989, filed on Jan. 24, 2012.

(51) Int. Cl. A61M 29/00

(52) U.S. Cl. CPC A61M 29/00
USPC 606/198

(57) ABSTRACT

Techniques are described for occluding the ostium of an appendage. In one example, an implantable medical device for insertion in a left atrial appendage of a patient includes a center hub having a longitudinal axis and a first side and a second side, a plurality of compression springs, each of the plurality of compression springs extending radially from the center hub, and a cover disposed about the compression springs and engaged to center hub on both the first side and the second side.

### CROSS REFERENCE TO RELATED APPLICATIONS

This application claims priority from U.S. Application No. 61/589,989, filed on Jan. 24, 2012, the entire contents of which is hereby incorporated by reference.

### TECHNICAL FIELD

The disclosure relates to implantable medical devices and, more particularly, to implantable medical devices that occlude appendages.

### BACKGROUND

The left atrial appendage (LAA) is a pouch-like extension of the left atrium of the heart. In some patients, blood clots form in the LAA. These blood clots may dislodge and enter the bloodstream, migrate through the anatomy, and block a vessel in the brain or heart, for example. A blocked vessel may cause cardiac arrhythmia, e.g., atrial fibrillation, which may lead to ischemic stroke.

Implanted medical devices are available for insertion into the ostium of the LAA to occlude the LAA and thus block blood clots from entering into the systemic circulation. In general, these devices are delivered to the LAA through a catheter system that enters the venous circulation, e.g., the inferior vena cava, and approaches the left atrium through the atrial septum between the right and left side of the heart, e.g., via a previously created hole in the atrial septum created using transseptal crossing techniques. The delivery catheter is guided through the septum toward the ostium of the LAA. After acquisition and insertion into the LAA, the implanted medical device is deployed so that it remains in the appendage. Once positioned, the implanted medical device is released by the catheter, and the catheter system is removed.

### SUMMARY

In general, this disclosure describes techniques for occluding the ostium of the left atrial appendage (LAA) of a heart. In some cases, a device that includes compression springs is positioned at the ostium of the LAA. A cover disposed about the device occludes the LAA and prevents blood clots from entering the blood stream. The device may be configured to ensure that any size and geometry of ostium of an LAA is occluded.

In one embodiment, this disclosure is directed to a device an implantable medical device for insertion in a left atrial appendage of a patient comprising a center hub having a longitudinal axis and a first side and a second side, a plurality of compression springs, each of the plurality of compression springs extending radially from the center hub, and a cover disposed about the compression springs and engaged to center hub on both the first side and the second side.

In another embodiment, this disclosure is directed to a method of implanting a medical device for insertion into a left atrial appendage. The method comprises providing an implantable medical device that comprises a center hub having a longitudinal axis and a first side and a second side, a plurality of compression springs, each of the plurality of compression springs extending radially from the center hub, and a cover disposed about the compression springs and engaged to center hub on both the first side and the second side. The method further includes collapsing the device within a deliver catheter and deploying the device at a deployment site by removing the device from the delivery catheter and allowing the compression springs to expand.

The details of one or more aspects of the disclosure are set forth in the accompanying drawings and the description below. Other features, objects, and advantages will be apparent from the description and drawings, and from the claims.

### BRIEF DESCRIPTION OF DRAWINGS

FIG. 1 is a perspective view of the distal side of an example implantable medical device, in accordance with this disclosure.

FIG. 2 is a perspective view of the proximal side of the example device depicted in FIG. 1, in accordance with this disclosure.

FIG. 3 is a conceptual diagram illustrating the device of FIG. 1 positioned at the ostium of the left atrial appendage, in accordance with this disclosure.

FIG. 4 is a side view of the example device depicted in FIG. 1, in accordance with this disclosure.

FIG. 5 is a front view of the example device depicted in FIG. 1, in accordance with this disclosure.

FIG. 6 is a side view of another example device, in accordance with this disclosure.

FIG. 7 is a perspective view of the example device depicted in FIG. 6.

FIG. 8 is a side view of another example device, in accordance with this disclosure.

FIG. 9 is a perspective view of the example device depicted in FIG. 8.

FIG. 10 is a front view of the example device depicted in FIGS. 8 and 9.

FIG. 11 is a flow diagram depicting an example method, in accordance with this disclosure.

### DETAILED DESCRIPTION

This disclosure describes techniques for occluding the ostium of an appendage, e.g., the left atrial appendage (LAA) of a heart. In some examples, the techniques may be effective in preventing cardiac arrhythmia, e.g., atrial fibrillation, by blocking blood clots formed in the LAA from entering the bloodstream. A vessel blocked by a blood clot may cause cardiac arrhythmia, e.g., atrial fibrillation, which may lead to ischemic stroke.

Left atrial appendage ostia have various sizes and geometries. As such, many existing LAA occluding devices are available in numerous sizes in order to accommodate the specific size of each patient's ostium. Currently, in some cases, prior to implantation of an LAA occluding device, a clinician estimates the size of a patient's ostium, e.g., using an average of measurements taken from several trans-esophageal echocardiogram images. Once the size of the patient's ostium is estimated, the clinician selects an occluding device having dimensions that will fit that patient's ostium. Hence, many differently-sized occluding devices must be manufactured, stocked, and available to the clinician in order to accommodate the variation in ostium sizes.

Although existing occluding devices are available in numerous sizes to accommodate variations in ostium size, the aforementioned existing occluding devices may not accommodate variations in ostium geometries between patients. That is, because of the irregularity of the shape of the ostium, many existing occluding devices may not conform entirely to the shape of a patient's ostium. As such, undesirable gaps may exist between the ostium wall and existing occluding devices.

In accordance with some example techniques of this disclosure, an implantable medical device is disclosed that can accommodate variations in both the size and geometry of the ostium of the LAA. In addition, after deployment the device may be easily recaptured and redeployed until the device properly seals off the LAA and prevents blood clots from entering the bloodstream.

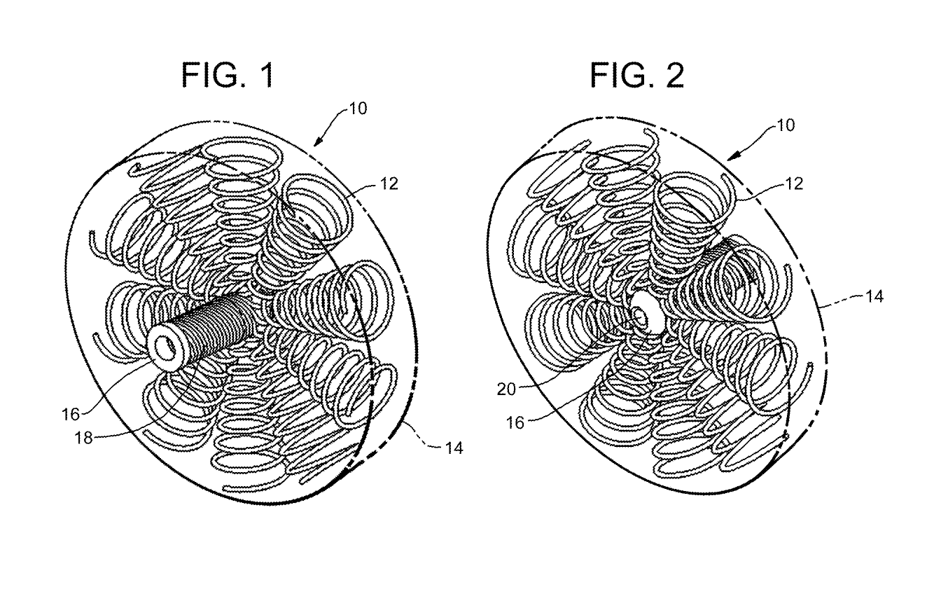

FIG. 1 is a perspective view of the distal side of an example implantable medical device, in accordance with this disclosure. In particular, FIG. 1 depicts an implantable medical device, shown generally at 10, which includes a plurality of compression springs 12 in an expanded state, and cover 14 disposed about springs 12 for occluding an appendage, e.g., LAA, of a patient. A compression spring is designed to operate with a compression load such that the spring becomes shorter as the load is applied to the spring. A compression spring has a longitudinal axis and has a plurality of turns disposed about the longitudinal axis.

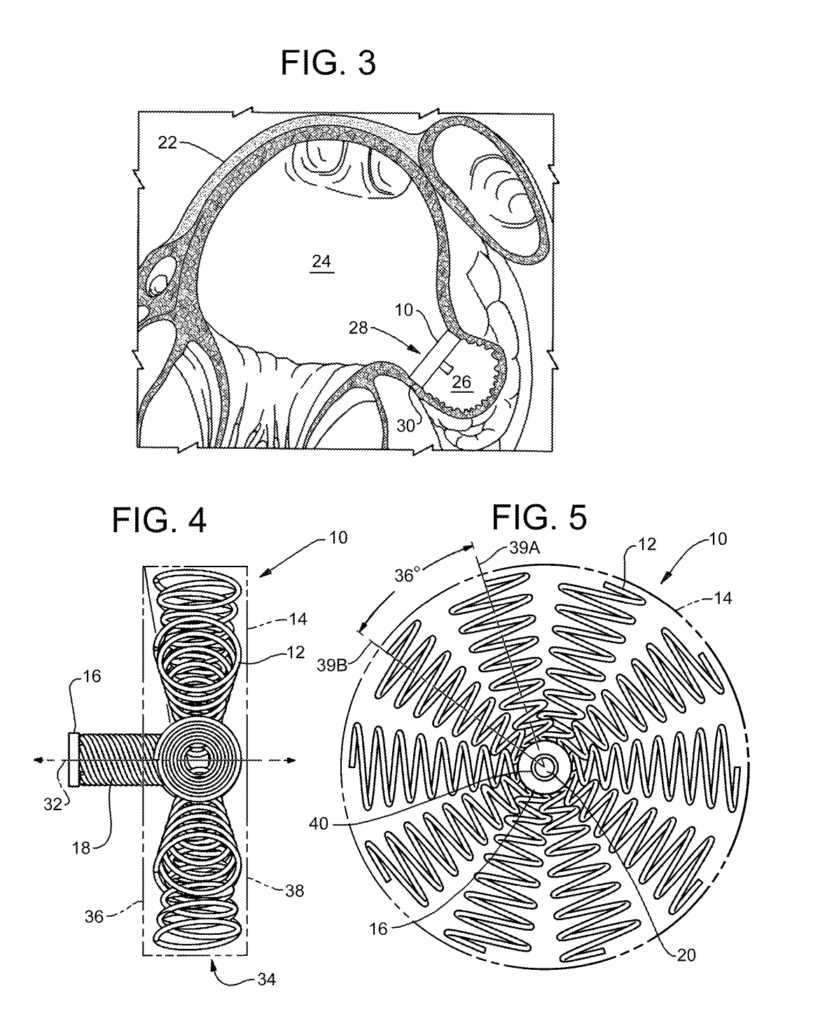

As shown and described in more detail below with respect to FIG. 3, device 10 is delivered and deployed at the ostium of the LAA, at which point device 10 and, in particular, springs 12 expand from a compressed state toward a deployed state. In the deployed state, springs 12 expand radially outward against portions of the ostium wall and to various extents from one another, resulting in a compression fit that seals off the LAA and prevents detached blood clots in the LAA from entering the bloodstream. That is, each of compression springs 12 compress against the ostium wall differently from one another, thereby allowing device 10 to conform to and sealingly engage an irregularly shaped ostium. In this manner, device 10 may adjust to the various sizes and shapes of ostia. Thus, device 10 may provide a better seal of the LAA than existing occluding devices. In addition, device 10 may eliminate the need for numerous differently sized devices by providing a design that can expand to accommodate the variation in ostium sizes.

As seen in the example depicted in FIG. 1, compression springs 12 may be conically shaped such that the widest portion of the cone presses against the ostium wall. A conical shape of compression springs 12 reduces or eliminates the opportunity for springs 12 to interfere with one another at the inner diameter of device 10 (near center hub 16) while still providing a sufficient amount of force against the ostium wall to secure the device at the ostium of the LAA.

In some examples, compression springs 12 are made of a biocompatible metal. In one example configuration, compression springs 12 are made of a shape-memory material such as a nickel-titanium alloy, e.g., nitinol. In another example, configuration, compression springs 12 are made of stainless spring steel.

As indicated above and in accordance with this disclosure, device 10 is designed to sealingly engage various sizes and geometries of ostia. In order to accommodate the various sizes and geometries of ostia, device 10 may have a diameter in the range of about 26 millimeter (mm) to about 36 mm. In one example configuration, device 10 may have a diameter of about 34 mm.

In addition, in one example configuration, device 10 should apply a force of about 200 grams against an ostium wall to provide a sufficient compression fit that will prevent device 10 from becoming dislodged. The spring constant of a compression spring will depend on the number of springs 12 in device 10. For example, device 10 of FIG. 1 includes ten compression springs. Therefore, for device 10 to apply about 200 grams of force against an ostium wall, each of the ten compression springs 12 should have a spring constant that is designed to apply a force of about 20 grams. Of course, device 10 may be designed to apply more, or less, force against an ostium wall than 200 grams.

In addition, it should be noted that device 10 of FIG. 1 is only one example configuration. In other example configurations, device 10 may include more than ten compression springs, e.g., 12 compression springs, and in yet other example configurations, device 10 may include less than ten compression springs, e.g., 8 compression springs. In configurations that include more than ten compression springs, each spring 12 will have a lower spring constant if device 10 is to apply the same amount of force as a device 10 with ten compression springs. Similarly, in configurations that include less than ten compression springs 12, each spring 12 will have a higher spring constant if device 10 is to apply the same amount of force as a device 10 with ten compression springs.

In some examples, each of springs 12 of device 10 has substantially the same spring constant. In other examples, one or more of springs 12 has a spring constant that is different from the spring constants of the other springs 12.

Each of springs 12 extend through center hub 16 of device 10. Upon exiting hub 16, one end of each of springs 12 are wrapped around hub 16 to form coil 18. Springs 12 may be secured by welding them to hub 16. In another example, springs 12 may be secured to the hub via a crimp ring, which is then welded to hub 16.

In order to occlude the LAA and thus block blood clots from entering the bloodstream, device 10 includes cover 14. Cover 14 attaches at hub 16 on both sides of device 10. Cover 14 is disposed about and fully encloses springs 12.

Cover 14 is made of a material that provides the desired permeability for an intended use. In some examples, cover 14 may block the passage of blood clots, but is permeable to blood flow therethrough, e.g., such as a filter. Alternatively, cover 14 can be of a material impermeable to blood flow. Cover 14 may be fabricated from any suitable biocompatible material such as, but not limited to, expanded polytetrafluoroethylene or ePFTE, (e.g., Gortex®), polyester, (e.g., Dacron®), PTFE (e.g., Teflon®), silicone, urethane, metal fibers, and other biocompatible polymers.

In some examples, at least a portion of one or more of springs 12 and/or cover 14 is configured to include one or more mechanisms for the delivery of a therapeutic agent. Often the agent will be in the form of a coating or other layer (or layers) of material placed on a surface region of the framework, which is adapted to be released at the site of the framework's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

It may be desirable to provide aspects of device 10 with the ability to safely biodegrade over time. Thus, in some examples, springs 12 and/or cover 14 is constructed from biodegradable materials that are also biocompatible. A biodegradable material is a material that will undergo breakdown or decomposition into harmless compounds as part of a normal biological process.

In one example configuration, device 10 may include one or more areas, bands, coatings, members, etc. that is (are) detectable by imaging modalities such as X-Ray, MRI, ultrasound, etc. In some examples, at least a portion of one or more of springs 12 or hub 16 is at least partially radiopaque.

FIG. 2 is a perspective view of the proximal side of the example device depicted in FIG. 1, in accordance with this disclosure. Hub 16 defines aperture 20 that allows hub 16 to attach to a delivery catheter (not depicted). Hub 16 extends from the proximal side of device 10 through to the distal side of device 10 to hold coil 18 in place.

FIG. 3 is a conceptual diagram illustrating the device of FIG. 1 positioned at the ostium of the left atrial appendage, in accordance with this disclosure. As seen in FIG. 3, heart 22 includes left atrium 24 and left atrial appendage 26. Positioned at the ostium of left atrial appendage 26, shown generally at 28, is occluding device 10. FIG. 3 depicts device 10 in a deployed state such that springs 12 pressed against various portions of ostium wall 30. In this manner, occluding device 10 sealingly engages ostium wall 30 via a compression fit, thereby preventing blood clots formed within LAA 26 from enter the bloodstream.

FIG. 4 is a side view of the example device depicted in FIG. 1, in accordance with this disclosure. In the example depicted in FIG. 4, device 10 has a longitudinal axis 32 and includes a single set of radially extending springs 12, shown generally at 34. Single set 34 of springs 12 is positioned at a first position along longitudinal axis 32. Hub 16 extends from first side 36 of device 10 to second side 38 of device 10.

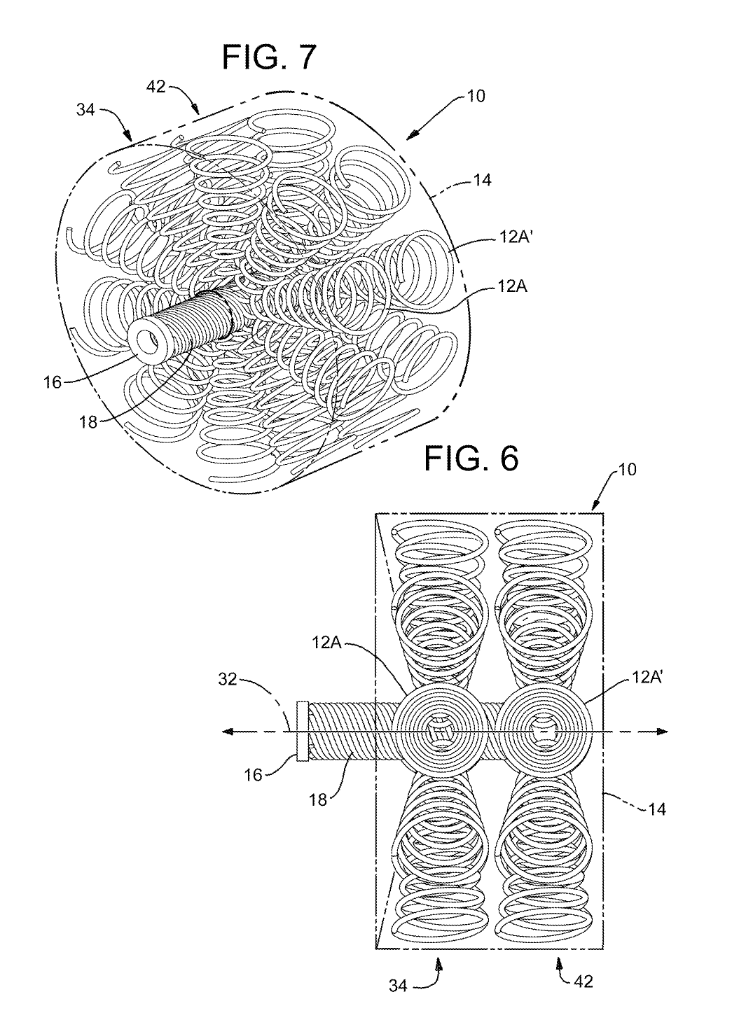

In other example configurations, as shown and described in more detail below with respect to FIGS. 6 and 7, device 10 may have two (or more) sets of springs 12 positioned at a first position and a second position along longitudinal axis 32.

FIG. 5 is a front view of the example device depicted in FIG. 1, in accordance with this disclosure. More particularly, FIG. 5 shows the ten compression springs 12 of device 10 spaced substantially equally apart from one another. That is, center lines extending through each of the ten springs 12, e.g., center lines 39A and 39B, are spaced apart from one another by at about 36 degrees (360 degrees/10 springs). Of course, for configurations with more, or fewer, equally spaced springs 12, the spacing between springs 12 will be different than 36 degrees. For example, a device with 12 springs that are spaced equally apart will have each spring spaced apart by about 30 degrees (360 degrees/12 springs).

It should be noted that in some example configurations, each of springs 12 are not spaced substantially equally apart from one another (not depicted). Rather, it may be desirable for two springs 12, for example, to be closer to one another than other pairs of springs 12.

Device 10 further includes connector 40. In one example, connector 40 is threaded to releaseably couple device 10 to a delivery catheter. Connector 40 allows device 10 to be deployed and recaptured and redeployed, if necessary. Of course, a threaded connector is only one specific example of connector. Other example connectors are considered within the scope of this disclosure. Device 10 is tethered to the delivery catheter via a deployment wire (not depicted). In some examples, the deployment wire has a screw on one end to engage connector 40.

FIG. 6 is a side view of another example device, in accordance with this disclosure. More particularly, in the example depicted in FIG. 6, device 10 has a longitudinal axis 32 and, in contrast to the example shown in FIG. 4, includes two sets of radially extending springs 12 along longitudinal axis 32, shown generally at 34, 42. First set 34 and second set 42 of springs 12 are positioned at a first position and a second position, respectively, along longitudinal axis 32.

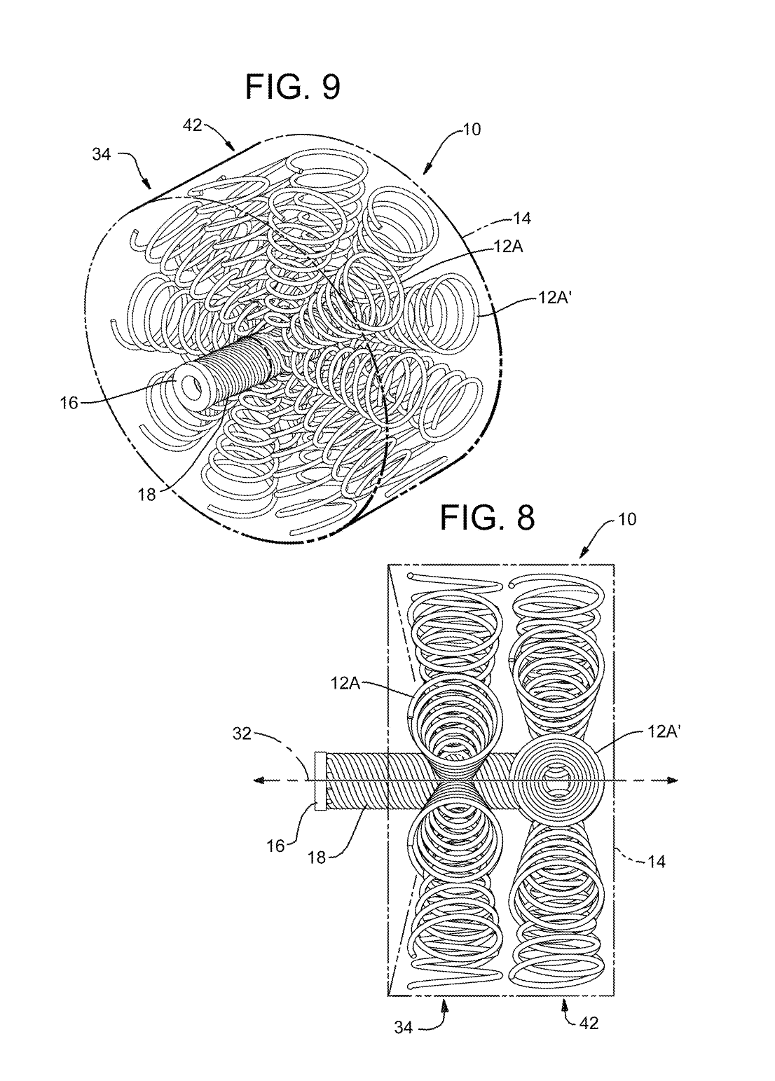

As seen in FIG. 6, the orientation of the two sets 34, 42 is substantially similar. That is, each spring 12 of first set 34 is substantially radially aligned with a corresponding spring 12 of second set 42, as viewed along longitudinal axis 32. As one example, spring 12A of set 34 is substantially radially aligned with corresponding spring 12A′ of set 42. In other example configurations, the orientation of the two sets 34, 42 is not substantially similar, as described in more detail below with respect to FIG. 8.

It should be noted that in some examples there may be more than two sets of springs 12. In addition, in one example configuration, one set of compression springs may have more or fewer springs than another set of springs 12. For example, set 34 of FIG. 6 may have ten compression springs 12 and set 42 may have eight compression springs 12. In such a configuration, the eight compression springs of set 42 may be aligned with eight of the ten springs of set 32, or configured in some other manner.

FIG. 7 is a perspective view of the example device depicted in FIG. 6. As described above with respect to FIG. 6, device 10 includes two sets of radially extending springs 12, shown generally at 34, 42.

FIG. 8 is a side view of another example device, in accordance with this disclosure. More particularly, in the example depicted in FIG. 8, device 10 has a longitudinal axis 32 and, in contrast to the example shown in FIG. 6, includes two sets of radially extending springs 12 along longitudinal axis 32, shown generally at 34, 42, that are offset from another. In FIG. 8, set 34 is not radially aligned with set 42 as viewed along longitudinal axis 32, in contrast to the example described above with respect to FIG. 6. Rather, sets 34, 42 are offset from one another such that each spring 12 of set 34 is not substantially radially aligned with a corresponding spring 12 of set 42. As one example, spring 12A of set 34 is offset from spring 12A′ of set 42.

In the particular example depicted in FIG. 8, device 10 has 20 compression springs 12: 10 springs in set 34 and 10 springs in set 42. The 10 springs in set 34 are spaced apart by about 36 degrees. Similarly, the 10 springs in set 42 are spaced apart by about 36 degrees. However, in the offset configuration depicted in FIG. 8, set 34 is oriented such that each spring in set 34 is offset from each spring in set 42 by 18 degrees. That is, a center line extending through a spring in set 34 is offset from a center line extending through a spring in set 42 by 18 degrees, thereby filling the 36 degree “gap” between adjacent springs in set 32. In other example configurations, the orientation of the two sets 34, 42 is not substantially similar.

It should be noted that in some examples there may be more than two sets of springs 12. In addition, in one example configuration, one set of compression springs may have more or fewer springs than another set of device 10. For example, set 34 of FIG. 8 may have ten compression springs 12 and set 42 may have eight compression springs 12. In such a configuration, the eight compression springs of set 42 may be offset from eight of the ten springs of set 34.

FIG. 9 is a perspective view of the example device depicted in FIG. 8. As described above with respect to FIG. 8, device 10 includes two sets of radially extending springs 12, shown generally at 34, 42.

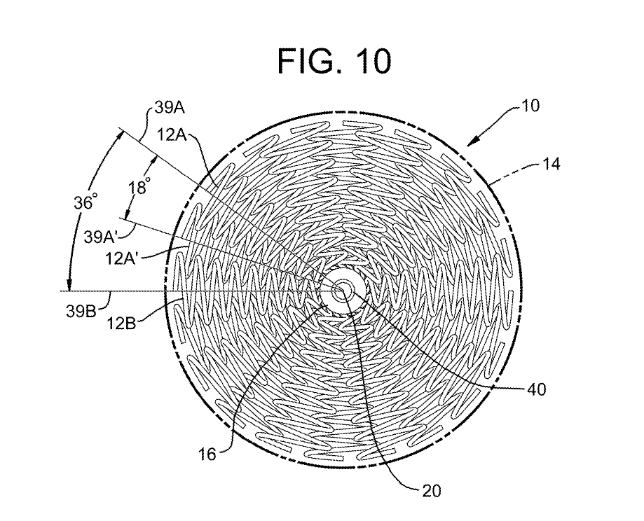

FIG. 10 is a front view of the example device depicted in FIGS. 8 and 9. More particularly, FIG. 10 shows two sets of radially extending springs 12. A center line, e.g., center line 39A, extending through a spring 12A in a first set of springs, e.g., set 34 of FIGS. 7 and 8, is offset by 36 degrees from a center line, e.g., center line 39B, extending through spring 12B in the first set of springs, and is also offset by 18 degrees from a center line, e.g., center line 39A′, extending through spring 12A′ in a second set of springs, e.g., set 42 in FIGS. 7 and 8. In this manner, the springs in one set fill the 36 degree “gap” between adjacent springs in the other set of springs.

To load device 10, a clinician, for example, collapses device 10 within a delivery catheter. Compression springs 12 of device 10 are bent proximally within the delivery catheter during delivery. In a partially deployed state, a portion of compression springs 12 remains within the delivery catheter during delivery. When device 10 is properly positioned and fully deployed, device 10 is untethered from the deployment wire. It should be noted that device 10 may also be preloaded into a delivery catheter by the device manufacturer.

During recapture by the delivery catheter, compression springs 12 are pulled proximally into the delivery catheter. Compression springs 12 of device 10 are bent distally as device 10 is recaptured by the delivery catheter.

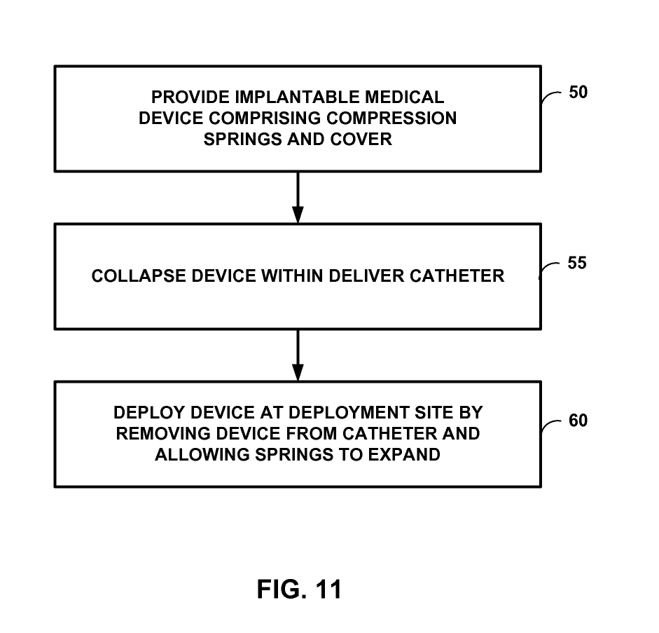

FIG. 11 is a flow diagram depicting an example method, in accordance with this disclosure. In the example method depicted in FIG. 11, an implantable medical device, e.g., device 10 of FIG. 4, for insertion into a left atrial appendage or, more particularly at the ostium of the LAA, is provided (50). Device 10 comprises center hub 16 having longitudinal axis 32 and first side 36 and second side 38, a plurality of compression springs 12, each of the plurality of compression springs 12 extending radially from center hub 16, and a cover 14 disposed about the compression springs and engaged to center hub 16 on both the first side and the second side. A clinician, for example, collapses device 10 within a delivery catheter (55). Finally, the clinician deploys device 10 at a deployment site, e.g., at the LAA, by removing device 10 from the delivery catheter and allowing compression springs 12 to expand (60). In a subsequent optional act, the clinician may recapture device 10 using the delivery catheter by retracting device 10 into the delivery catheter, e.g., using a deployment wire tethered between the delivery catheter and device 10. In another subsequent optional act to recapture, the clinician may redeploy device 10, e.g., at the LAA, by removing device 10 from the delivery catheter and allowing compression springs 12 to expand (60).

Various aspects of the disclosure have been described. These and other aspects are within the scope of the following claims.

1. An implantable medical device for insertion in a left atrial appendage of a patient comprising:
a center hub having a longitudinal axis and a first side and a second side;
a plurality of compression springs, each of the plurality of compression springs extending radially from the center hub; and
a cover disposed about the compression springs and engaged to center hub on both the first side and the second side.

2. The device of claim 1, wherein each of the plurality of compression springs is conically shaped.

3. The device of claim 1, wherein the plurality of compression springs are arranged in a first set, and wherein the first set is positioned at a first position along the longitudinal axis.

4. The device of claim 1, wherein the plurality of compression springs are arranged in at least a first set and a second set, wherein the first set is positioned at a first position along the longitudinal axis, wherein the second set is positioned at a second position along the longitudinal axis.

5. The device of claim 4, wherein the first set and the second set comprise an equal number of compression springs.

6. The device of claim 4, wherein the first set and the second set comprise an unequal number of compression springs.

7. The device of claim 5, wherein each spring of the first set is substantially radially aligned with a spring of the second set as viewed along the longitudinal axis.

8. The device of claim 5, wherein each spring of the first set is radially offset from a spring of the second set as viewed along the longitudinal axis.

9. The device of claim 1, wherein at least a portion of the device comprises a therapeutic agent.

10. The device of claim 9, wherein the at least one therapeutic agent is a coating selected from at least one member of the group consisting of non-genetic therapeutic agents, genetic therapeutic agents, cellular material, and any combination thereof.

11. The device of claim 10, wherein the coating is disposed about at least a portion of the cover.

12. The device of claim 10, wherein the coating is disposed about at least a portion of one of the springs.

13. The device of claim 1, wherein at least a portion of the device is radiopaque.

14. The device of claim 1, wherein at least a portion of the device is biodegradable.

15. A method of implanting a medical device for insertion into a left atrial appendage, the method comprising:
providing an implantable medical device comprising:
a center hub having a longitudinal axis and a first side and a second side;
a plurality of compression springs, each of the plurality of compression springs extending radially from the center hub; and
a cover disposed about the compression springs and engaged to center hub on both the first side and the second side;
collapsing the device within a deliver catheter; and
deploying the device at a deployment site by removing the device from the delivery catheter and allowing the compression springs to expand.
